(19) United States
(12) Patent Application Publication
(10) Pub. No.: US 2018/0180633 A1
(43) Pub. Date: Jun. 28, 2018

(54) METHODS OF RATIONALLY DESIGNING A PERSONALIZED NUTRITIONAL PLAN

(71) Applicant: Virta Health Corp., San Francisco, CA (US)

(72) Inventors: Jeffrey Scott Volek, Dublin, OH (US); Stephen Dodge Phinney, Elk Grove, CA (US)

(21) Appl. No.: 15/851,997

(22) Filed: Dec. 22, 2017

Related U.S. Application Data
(60) Provisional application No. 62/438,760, filed on Dec. 23, 2016.

(51) Int. Cl. G01N 33/92; A23L 33/00; A23L 33/125

(52) U.S. Cl. CPC G01N 33/92; A23L 33/30; A23V 2002/00; G01N 2800/042; G01N 2800/52; A23L 33/125

(57) ABSTRACT

Provided herein are methods of rationally designing a personalized nutritional plan.

### CROSS REFERENCE TO RELATED APPLICATIONS

This application claims the benefit of priority under 35 U.S.C. § 119(e) to U.S. Application No. 62/438,760 filed on Dec. 23, 2016, which is incorporated herein in its entirety.

### TECHNICAL FIELD

This disclosure generally relates to methods of rationally designing a personalized nutritional plan.

### BACKGROUND

It has been demonstrated that individuals respond very differently to the food they eat. However, the vast majority of diets and nutritional programs do not take into consideration an individual's unique physiology, metabolism and reactions to dietary intake, particularly as the individual changes over the course of a life time. For example, ketogenic diets emphasize restricting carbohydrate intake. Ketogenic diets that limit dietary carbohydrate result in a greater reliance on fatty acids and ketones for energy, and this shift in metabolic fuel use is associated with greater ease of fat loss and a number of favorable health outcomes (e.g., reduced food intake due to feeling satiated, improved fuel flow to the brain) resulting from the physiological levels of ketones produced as a result of restricting carbohydrate intake.

However, even successful diets rely upon brute force, one-size-fits-all approaches. For example, in the popular Atkins diet, carbohydrate-containing foods are all but eliminated in the early stages of the regimen. This uniform and severe degree of carbohydrate restriction is necessitated by the lack of accurate, personalized and physiologically-derived indicators of an individual's response to a reduction or a near-complete elimination of carbohydrates from the diet. Unfortunately, such severe measures also tend to discourage people from continuing the diet.

### SUMMARY

In one aspect, a method of determining a healthy carbohydrate intake for an individual is provided. Such a method typically includes a) establishing nutritional ketosis in the individual; b) determining, while the individual is experiencing nutritional ketosis, the amounts of one or more fatty acids in a biological sample from the individual to generate an optimal range of one or more fatty acids, wherein the one or more fatty acids are selected from palmitoleic acid (POA) and di-homo gamma-linolenic acid (DGLA); and c) prescribing a diet comprising appropriate carbohydrates, or appropriate carbohydrates and proteins, such that the amounts of the one or more fatty acids are maintained within the optimal range. In some embodiments, appropriate carbohydrates includes an appropriate amount of carbohydrates and/or an appropriate glycemic index of carbohydrates.

In another aspect, a method of determining an upper limit of healthy carbohydrate intake for an individual is provided. Such a method typically includes prescribing a diet to the individual that comprises a healthy carbohydrate intake, wherein the healthy carbohydrate intake is determined for the individual by: establishing nutritional ketosis in the individual; determining, while the individual is experiencing nutritional ketosis, the amounts of one or more fatty acids in a biological sample from the individual to generate an optimal range of the one or more fatty acids, wherein the one or more fatty acids are selected from palmitoleic acid (POA) and di-homo gamma-linolenic acid (DGLA). Generally, a diet that includes a healthy carbohydrate intake is a diet that maintains the amounts of the one or more fatty acids within the optimal range. In some embodiments, healthy carbohydrate intake includes a healthy amount of carbohydrate intake and/or a healthy glycemic index of carbohydrates.

In some embodiments, the individual is at risk of developing pre-diabetes or diabetes. In some embodiments, the individual has been diagnosed with pre-diabetes or diabetes. In some embodiments, the individual is being treated for type-2 diabetes with a medicament. In some embodiments, the individual has been diagnosed with a disorder associated with insulin resistance. Representative disorders associated with insulin resistance include, without limitation, obesity, metabolic syndrome, hypertension, hepatic steatosis, polycystic ovary syndrome, and sleep apnea. In some embodiments, nutritional ketosis is established when the concentration of ketones is about 0.4 mM to about 4 mM. In some embodiments, nutritional ketosis is established when the concentration of ketones is about 0.5 mM to about 3 mM. In some embodiments, nutritional ketosis is established when the concentration of ketones is at least about 0.5 mM. In some embodiments, nutritional ketosis is established when the concentration of ketones is at least about 0.75 mM. In some embodiments, nutritional ketosis is established when the concentration of ketones is at least about 1.0 mM. In some embodiments, the concentration of ketones is determined in a biological sample selected from whole blood, plasma, serum, urine, tears, and breath. In some embodiments, nutritional ketosis is maintained in the individual for at least about a week before the determining step is performed. In some embodiments, nutritional ketosis is maintained in the individual for at least about two weeks before the determining step is performed. In some embodiments, nutritional ketosis is maintained in the individual for at least about one month before the determining step is performed.

In some embodiments, the at least one biological sample is a buccal swab, In some embodiments, the at least one biological sample comprises cheek cells. In some embodiments, the at least one biological sample is whole blood, red blood cells, plasma, or serum (e.g., serum phospholipids, serum cholesteryl esters, serum triglycerides).

In some embodiments, the determining step is qualitative. In some embodiments, the method further includes determining the amounts of one or more fatty acids in a biological sample from the individual when the individual is not experiencing nutritional ketosis. In some embodiments, the determining step is repeated.

In some embodiments, the method further includes modifying the diet of the individual so as to maintain the one or more fatty acids within the optimal range. In some embodiments, the method further includes adjusting the amount and/or glycemic index of carbohydrates consumed by the individual so as to maintain the one or more fatty acids within the optimal range. In some embodiments, the method further includes reducing the protein intake of the individual, if necessary, so as to maintain the one or more fatty acids within the optimal range. In some embodiments, the method further includes determining a baseline of the one or more fatty acids prior to establishing nutritional ketosis. In some embodiments, the method further includes analyzing serum and cheek cell data of the individual to further refine the optimum ranges for the at least one fatty acid to predict long-term weight stability.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only, and not intended to be limiting. All publications, patent applications, patents, and other references mentioned herein are incorporated by reference in their entirety.

### DESCRIPTION OF DRAWINGS

FIG. 1 shows the chemical structure of palmitoleic acid (POA).

FIG. 2 shows the chemical structure of dihomo-gamma-linolenic acid (DGLA).

FIG. 3 is a representative graph that illustrates the response by multiple individuals to carbohydrate intake as measured by fatty acid levels.

FIG. 4 is a representative graph that illustrates the response of an individual to protein intake under low carbohydrate conditions based on fatty acid levels.

### DETAILED DESCRIPTION

The present disclosure provides methods to generate individualized-specific dietary guidance to manage the risk of a pathology such as diabetes or obesity. Such methods enable an individual to objectively tailor their dietary intake, primarily of carbohydrates, in response to physiological changes in order to effectuate a healthy weight and/or prevent, minimize the effect of, or reverse diseases such as diabetes or pre-diabetes. The methods provided herein are based on the particular carbohydrate-tolerance or, conversely, carbohydrate-intolerance, exhibited by an individual (see, for example, Phinney & Volek (2012, The Art and Science of Low Carbohydrate Performance, Beyond Obesity LLC) and Volek & Phinney (2011, The Art and Science of Low Carbohydrate Living: An Expert Guide to Making the Life-Saving Benefits Carbohydrate Restriction Sustainable and Enjoyable, Beyond Obesity LLC)), which can be used to design a personalized nutritional plan.

Methods that allow a rationally designed personalized nutritional plan are useful for individuals diagnosed with diabetes or pre-diabetes (or at risk of developing diabetes or pre-diabetes) and can be used to minimize the effects of type-2 diabetes. Such methods and the resulting personalized nutritional plans can be used to prevent or reverse type-2 diabetes or pre-diabetes, or to prevent recurrence of type-2 diabetes once it is in remission. The methods described herein also can be used, for example, to allow an individual to maximize or “fine tune” their carbohydrate intake within healthy limits (e.g., in the absence of increasing weight, to prevent re-gain of weight, to promote maintenance of weight loss, and/or to maintain a desired weight in an individual). Ultimately, the methods described herein can be used to treat or prevent diabetes, pre-diabetes, obesity, metabolic syndromes, hypertension, hepatic steatosis, polycystic ovary disease or other diseases associated with insulin resistance (e.g., Alzheimer's disease, and many fowls of cancer or chronic diseases). As used herein, “treating” refers to reduction, amelioration or mitigation of one or more disease symptoms. As used herein, “preventing” refers to a delay in the onset or a complete absence of the onset of one or more symptoms.

The methods described herein are based on establishing (i.e., generating, determining) an optimal range of at least one fatty acid for an individual, and then prescribing (i.e., recommending, imposing, requiring) an appropriate diet (e.g., one that contains an appropriate amount of carbohydrates) such that at least one fatty acid is maintained within the optimal range established for that individual. As described herein, the optimal range of at least one fatty acid is determined relative to an individual's specific carbohydrate tolerance/intolerance threshold. It would be understood that insulin resistance is closely linked to an impaired ability to manage blood glucose and, thus, exhibits as a form of carbohydrate intolerance.

The carbohydrate tolerance/intolerance threshold for an individual is determined by establishing nutritional ketosis in the individual and determining the level of at least one fatty acid, Methods of establishing nutritional ketosis in an individual are known in the art, and typically include eliminating nearly all carbohydrates from the individual's diet for a period of time (e.g., sufficient for ketoadaptation to occur). Depending on the particular individual, it can take about 2 weeks or more of carbohydrate restriction or elimination to establish nutritional ketosis (e.g., at least about 3 weeks, at least a month or more of carbohydrate restriction or elimination). Nutritional ketosis has been established in an individual when their serum or capillary blood beta-hydroxybutyrate (BHB) concentration is maintained between about 0.4 mM and about 4.0 m.M (e.g., between about 0.5 mM and about 3.5 mM, about 1.0 mM and about 3.0 mM, about 1.0 mM and about 2.0 mM; about 0.5 mM, about 1.0 mM, about 1,5 mM, about 2.0 mM, about 2.5 mM, about 3.0 mM, about 3.5 mM, or about 4.0 mM) for a period of time (e.g., at least 5 days, 7 days, 10 days, 14 days or more). The concentration of serum BHB can be determined using methods known in the art. For example, serum BHB can be quantified in the blood of an individual using enzymatic assays or indirectly in the breath of an individual (via the levels of acetone) using a breathalyzer. The concentration of ketones (e.g., BHB) also can be determined in biological samples including, without limitation, whole blood, plasma, urine, and tears.

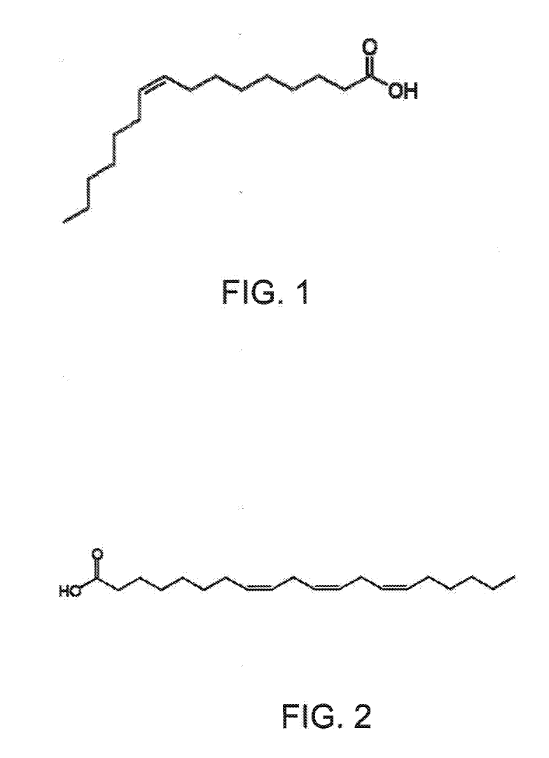

Once nutritional ketosis has been established in the individual, the level of at least one fatty acid is determined. One fatty acid that is suitable for use in the methods is palmitoleic acid (POA). FIG. 1 shows the chemical structure of POA. Palmitoleic acid, or (Z)-9hexadecenoic acid, is an omega-7 monounsaturated fatty acid having the formula CH₃(CH₂)₅CH=CH(CH₂)₇COOH, and is a component of the glycerides in human adipose tissue. POA is synthesized in the liver, primarily from carbohydrate substrates, with the last step being the production of POA from palmitic acid by the action of the enzyme delta-9 desaturase. POA is an indicator of the conversion of carbohydrates into fat, and POA levels increase when the body cannot immediately burn (as glucose) or store (as glycogen) all of the carbohydrates being ingested by an individual, Therefore, POA is an early indicator that the body is struggling to handle the amount and/or glycemic index of carbohydrates being consumed. Until now, however, POA has not been determined in a prospective manner and carbohydrate ingestion adjusted accordingly with the intent of keeping POA levels within a pre-determined range.

Another fatty acid that is suitable for use in the methods is di-horn gamma-linolenic acid (DGLA), also known as 8,11,14-eicosatrienoic acid. FIG. 2 shows the chemical structure of dihomo-gamma-linolenic acid. Dihomo-gamma-linolenic acid (DGLA) is a 20-carbon fatty acid with three cis double bonds. DGLA is the elongation product of gamma-linolenic acid (GLA; 18:3n-6), which, in turn, is a desaturation product of linoleic acid (18:2n-6). Like POA, DGLA is an early indicator that the body is struggling to efficiently metabolize the current level of carbohydrate intake. Unlike POA, however, DGLA is not a by-product of carbohydrate metabolism; instead, DGLA is an intermediate product in the omega-6 anabolic pathway leading to arachidonic acid (AA, 20:4n-6). Arachidonic acid is an important regulator of genes controlling lipogenesis, but also is highly vulnerable to destruction by reactive oxygen species (ROS, or oxygen free radicals). When an amount or glycemic index of dietary carbohydrate is consumed that goes beyond an individual's tolerance, ROS production increases, AA is destroyed, and blood and tissue levels of DGLA increase as the omega-6 anabolic pathway accelerates AA production.

As demonstrated herein, POA levels reflect the conversion of carbohydrate to fat, while DGLA levels reflect an aspect of fatty acid composition (i.e., stress on omega-6 essential fatty acid metabolism). What makes POA and DGLA such powerful tools as biomarkers is that two individuals, consuming the same diet, and generally having the same level of activity, may have very different responses to the same carbohydrate intake. Some individuals, through their unique physiology, are more tolerant to carbohydrates, whereas others may be far more sensitive (e.g., exhibit carbohydrate intolerance). The optimal range of one or more fatty acids can be used as a form of “map” or “standard curve,” representative of the carbohydrate tolerance/intolerance for the specific individual. The optimal range of one or more fatty' acids as described herein allows for the amount and/or glycemic index of carbohydrates in a diet to be “calibrated”. For example, adopting a “low carb” diet may be unnecessarily strict for some individuals and insufficient for others to effectuate weight loss, disease prevention, or other desirable outcomes. It would be understood that, for those individuals who can tolerate carbohydrates well, restriction of carbohydrate intake may not be required for attenuating or maintaining weight loss.

In some embodiments, the level of POA and/or DGLA is/are determined. Given the relationship between DGLA and arachidonic acid (ARA) in the omega-6 anabolic pathway, it would be understood that similar information could be obtained about an individual's carbohydrate intolerance by determining the levels of POA and/or ARA. Similarly, it would be appreciated by a skilled artisan that the levels of POA and/or DGLA (and/or ARA) can be determined directly, or the levels of POA and/or DGLA (and/or ARA) can be determined indirectly (e.g., based on one or more upstream or downstream fatty acids or intermediates in the biosynthetic pathway of POA and/or DGLA).

An individual's carbohydrate tolerance also or alternatively can be evaluated by measuring the amount of fatty acid(s) in the individual at an early age, at a healthy weight, and/or prior to the onset of disease. In addition to, or in lieu of, the methods described herein for determining an individual's specific carbohydrate tolerance, an individual's diet can be recorded in detail, for example, in a food intake diary, and the levels of at least one fatty acid can be continuously monitored. The pattern of increases and decreases in the levels of the at least one fatty acid can be correlated with the amount and/or glycemic index of carbohydrates in the diet. It would be appreciated that an individual's carbohydrate tolerance/intolerance, using any of the methods described herein, can be evaluated or determined more than once for an individual (e.g., at different ages).

Once the optimal levels of one or more fatty acids have been determined (e.g., while an individual is experiencing nutritional ketosis), those optimal levels can be used to design a diet for that individual that includes an appropriate amount and/or glycemic index of carbohydrates and also to monitor the individual for carbohydrate tolerance/intolerance once carbohydrates have been introduced back into the diet. As used herein, an appropriate amount and/or glycemic index of carbohydrates is an amount and/or glycemic index of carbohydrates in the diet of the individual that maintains the fatty acid(s) within the optimal range determined for that individual. For purposes of monitoring an individual's fatty acid levels, particularly once carbohydrates have been introduced back into the diet, it may be desirable to determine the fatty acid levels at a particular frequency (e.g., once a week, once a month, once every 6 months, once a year) or upon changes in the individual's health (e.g., a weight change, a diagnosis of a disease).

It would be understood that different carbohydrates (or carbohydrate-containing foods) are absorbed by a body at different rates. Glycemic index (GI) is an indication of how rapid a carbohydrate (or a carbohydrate-containing food) raises blood glucose (relative to a reference food, e.g., glucose or white bread; usually evaluated over a period of about 2 hours). Carbohydrates (or carbohydrate-containing foods) that break down quickly during digestion have a higher GI, while carbohydrates (or carbohydrate-containing foods) that break down slowly during digestion have a low GI. Simply by way of example, and without limitation, examples of carbohydrates considered to have a low glycemic index (e.g., a GI of about 55 or less) include 100% stone-ground whole wheat bread, pumpernickel bread, rolled or steel-cut oatmeal, oat bran, pasta, barley, bulgar, sweet potato, corn, peas, legumes and lentils, non-starchy vegetables, carrots, and most fruits; examples of carbohydrates considered to have a medium glycemic index (e.g., a GI of about 56 to about 69) include brown rice, wild rice, couscous, whole wheat bread, rye bread, pita bread, and quick oats; and examples of carbohydrates considered to have a high glycemic index (e.g., a GI of about 70 or more) include white bread, bagels, corn flakes, bran flakes, instant oatmeal, white rice, rice pasta, russet potato, pumpkin, pretzels, puffed rice, rice cakes, popcorn, saltine crackers, melons and pineapple. Adapted from the American Diabetes Association (diabetes.org on the World Wide Web).

The biological sample used to determine the level of the fatty acid(s) can be, without limitation, whole blood, red blood cells, plasma, serum (e.g., serum phospholipids, serum cholesteryl esters, serum triglycerides), or cheek cells. In some embodiments, the biological sample is obtained during routine laboratory work under the direction of a physician; in some embodiments, the biological sample is obtained by the individual as part of, for example, routine self-monitoring. It would be understood that the particular biological sample used in the methods described herein should be representative or reflective of the recent fatty acid levels in an individual's body. For example, while the biological sample for detecting the at least one fatty acid can be fat tissue, this is a longer lived tissue, which may not always be reflective of short term changes in diet and/or metabolism (in addition to being more invasive to collect). Therefore, in some embodiments, blood or other shorter lived tissues (e.g., cheek cells) often are preferred for evaluating short term changes and responses (e.g., to diet). It would he appreciated that cheek cells can be obtained using a buccal swab and can reflect changes (e.g., small changes) in fatty acids over a short period of time (e.g., days to weeks).

When the biological sample is blood, it can be obtained by a finger stick or a hypodermic phlebotomy. For example, a drop of blood obtained by a finger stick can be adsorbed onto filter paper, extracted by the method of Bligh/Dyer, trans-methylating the lipid soluble compounds (e.g., with sulfuric acid in methanol), followed by gas chromatography analysis. For blood obtained in a larger volume, the plasma can be separated, extracted by the method of Bligh/Dyer, and the phospholipids, triglycerides, and cholesterol esters can be separated by thin-layer chromatography. These three fractions then can be separately trans-esterified (e.g., using sulfuric acid in methanol), and the resulting fatty acid methyl esters can be quantitated using gas chromatography in order to determine levels of the particular fatty acid(s) of interest. In some embodiments, the optimal levels of one or more fatty acids can be determined (i.e., while an individual is experiencing nutritional ketosis) using blood as described herein under the direction of a physician.

Methods of fatty acid extraction, separation, and gas chromatography analysis for determining levels of fatty acids are known in the art, and it is within the skill of a person in the art to modify sampling and fatty acid characterization protocols as necessary and appropriate. Further, there are a number of other established methodologies for determining fatty acid levels within tissue samples including, but not limited to, methanol precipitation followed by gas chromatography, other HPLC techniques, and mass spectroscopy. All such methods of sampling and analyzing the levels of fatty acids are within the skill of the ordinary artisan.

As indicated herein, cheek cells also are well suited as a biological sample since they can be readily collected without any discomfort, they can be collected by the individual at their home, and cheek cells replace themselves every few days which ensures that the cells reflect the individual's current diet and metabolism. In addition, cheek cells are highly representative of serum fatty acid levels. For example, the cheek cells can be collected by a simple swabbing of the inside of the mouth. This may be performed by the individual in their own home, and the swab may be sent to a laboratory for analysis. Alternatively, the swab may provide a read-out (e.g., in the form of a symbol (e.g. “+” or “−”) or a color; see, for example, below) to the individual as an indication of their fatty acid levels and where those levels are relative to the optimal range determined for that individual.

Simply by way of example, the optimal range of the at least one fatty acid can be defined as 120% or less of a baseline value determined during nutritional ketosis. In one embodiment, the optimal range of the fatty acid(s) (e.g., less than 120% of the baseline value) can be reflected by a green color on an assay, a less-than-optimal range of the fatty acid(s) (e.g., between 120% and 140% of the baseline value) can be reflected by a yellow color on an assay, and a dangerous level of the fatty acid(s) (e.g., between 140% and 160%; or greater than 160%) can be reflected by a red color on an assay. Alternatively, a baseline value of at least one fatty acid can be determined prior to establishing nutritional ketosis and the optimal range of the fatty acid can be defined as, for example, not exceeding the nutritional ketosis value plus 20% of the difference between the baseline and the nutritional ketosis value.

It would be appreciated that, once the optimal range of the at least one fatty acid is determined for an individual, the absolute amount of the at least one fatty acid is not as critical as simply confirming that the level of the at least one fatty acid is maintained in, or at least near, the optimal range. Thus, as an alternative to the color coding system described herein, “+” and “-” symbols or a numerical system (e.g., “−1”, “0”, and “+1”) could be used.

The methods described herein can be performed on individuals who are susceptible to, or have been diagnosed with, diabetes or pre-diabetes. The methods described herein also can be performed (or continued to be performed) on individuals whose diabetes is in remission. Alternatively, the methods described herein are performed on individuals who are overweight or obese, or who are underweight due to, for example, malnourishment. The individuals referred to herein typically are human individuals, although the individuals referred to herein also can be animal individuals (e.g., companion animals, farm animals, exotic animals).

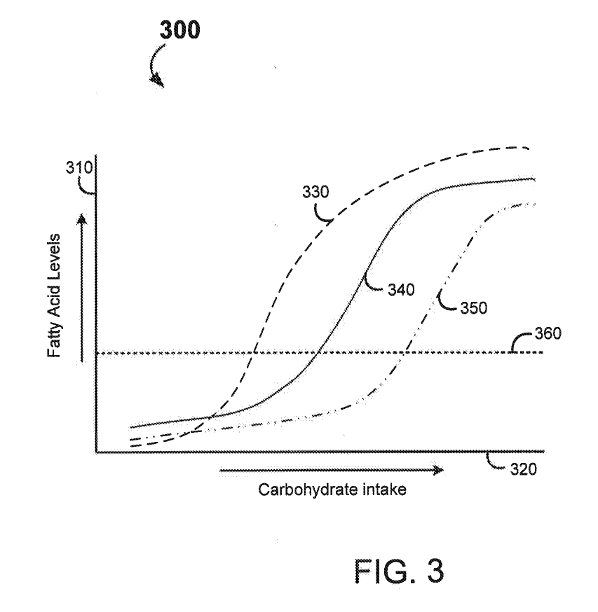

FIG. 3 is an exemplary graph 300 showing three hypothetical individuals' response (fatty acid level 310) to carbohydrate intake 320. The first individual, depicted as the plotted line 330, has a more sensitive reaction to carbohydrates than the others. As carbohydrate intake increases, this first individual rapidly begins to show signs of carbohydrate intolerance, thereby causing the level of fatty acids in that individual to increase rapidly. The second individual has a more muted response to increased carbohydrate intake, as depicted in plot line 340, and the third individual is relatively tolerant to increased carbohydrate intake, as depicted in plot line 350. The line 360 shown in FIG. 3 is demonstrating each individual's specific carbohydrate tolerance. Thus, according to the graph shown in FIG. 3, the first individual will require a far more carbohydrate restricted diet as compared to the second or third individual, in order to maintain the same efficacy of the diet. Likewise, the third individual has the greatest leniency in their carbohydrate intake to achieve similar results.

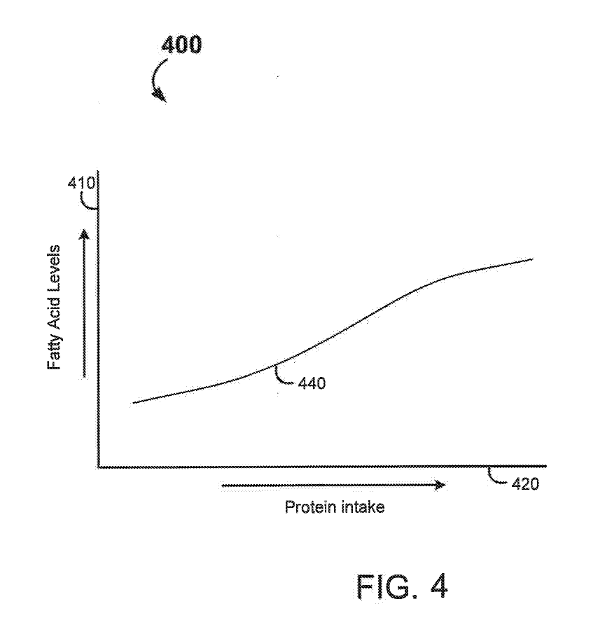

In addition to carbohydrate intake, POA and DGLA also can be influenced by protein intake. FIG. 4 is a representative graph showing the correlation between fatty acid levels and protein intake in a hypothetical individual who is already on a restricted carbohydrate diet. As indicated herein, each individual has a unique physiology that causes the shape of their response curves to differ from that of another individual. In the context of a low carbohydrate ketogenic diet, increasing levels of dietary protein increases serum insulin, which is a signal for increased lipogenesis. In the example graph 400, two axes are provided: the level of protein intake 420 and the level of the biomarker 410. As with FIG. 3, the biomarker can be a fatty acid such as POA and/or DGLA. The curve 440 shows this individual's biomarker response to increasing protein consumption while restricting carbohydrate intake.

If POA and/or DGLA levels continue to remain higher than desired, even when carbohydrates have been restricted, the amount of protein in the individual's diet can be reduced (i.e., in addition to carbohydrate restriction). It would be appreciated that these fatty acid levels are lowest when an individual is consuming a low carbohydrate and low protein diet. As protein intake increases, the level of the fatty acid(s) increases, but at a slower pace than if carbohydrate intake were to increase. In some instances, ingesting a low carbohydrate diet and/or a low protein diet may cause headaches, and individuals may benefit from adding a small amount of salt or sodium to the diet.

Since every individual has differing sensitivities to carbohydrate intake and to protein intake, the present disclosure allows each individual to tailor their dietary intake of carbohydrates and proteins, as well as fats, in order to ensure maximal efficacy of their diet. Therefore, the methods described herein can be used to provide an objective form of dietary guidance. Such methods enable individuals to tailor their diets to their carbohydrate tolerance levels, and more effectively reach and to sustain their dietary goals. In addition, the methods described herein can be employed by physicians to generate dietary guidance so as to treat, prevent or reverse diseases such as diabetes (e.g., type-2 diabetes) or pre-diabetes.

Based on the levels of the one or more fatty acids, the individual can be determined to be experiencing carbohydrate tolerance (e.g., associated with ingestion of an appropriate or healthy level of carbohydrates) or carbohydrate intolerance (e.g., associated with over-consumption of carbohydrates). Carbohydrate intolerance in an individual that is maintained for any period of time (e.g., days, weeks, months, years) can indicate, or be predictive of, the onset of diabetes or pre-diabetes. Accordingly, the methods described herein can include a subsequent treatment. This treatment can include, for example, instructing the individual to reduce or further reduce their intake of carbohydrates or adjusting the amount and/or glycemic index of carbohydrates consumed by the individual. It would be understood by the skilled artisan that, depending on the specific individual, adjusting can refer to increasing the amount and/or glycemic index of carbohydrates consumed by the individual or decreasing the amount and/or glycemic index of carbohydrates consumed by the individual. Additionally or alternatively, a nutritional plan can be generated for the individual based on the levels of the fatty acid(s); as described herein, a nutritional plan provides the individual with a suitable amount and/or glycemic index of carbohydrates to achieve the desired result (e.g., weight maintenance or loss, disease treatment, prevention, and/or reversal).

The methods described herein can be repeated as often as necessary to re-calibrate or re-evaluate a particular diet for an individual. For example, such methods can be repeated based on age or calendar milestones (e.g., at 30, 50, 60, etc. years of age; yearly or every five or ten years) or based on changes in one or more health parameters of an individual (e.g., weight gain, progression of disease (e.g., pre-diabetes to diabetes)), It would be appreciated that individuals undergo changes (e.g., metabolic changes) as they age, ingest different diets, live in different geographical areas, experience the onset and/or remission of disease (e.g., cancer), such that an individual's carbohydrate tolerance/intolerance threshold may change. Therefore, the methods described herein using an individual's optimal ranges for a fatty acid can be used to monitor the individual's carbohydrate tolerance (or intolerance, as the case may be) and adjust the carbohydrate levels accordingly so as to provide a healthy diet over their lifetime.

The methods described herein can be used to determine an appropriate amount of carbohydrate intake for an individual in order for them to lose weight, maintain weight, or gain weight. In some instances, the methods described herein are performed on an individual who has been identified as at risk of developing pre-diabetes or diabetes, or who has been diagnosed with pre-diabetes or diabetes. Methods of identifying an individual who is at risk of developing pre-diabetes or diabetes are known in the art, and methods of diagnosing pre-diabetes and diabetes also are known in the art. In addition, the methods described herein also can be used to complement the use of nutritional ketosis in an individual.

In accordance with the present invention, there may be employed conventional molecular biology, microbiology, biochemical, and recombinant DNA techniques within the skill of the art, Such techniques are explained fully in the literature.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of determining a healthy carbohydrate intake for an individual, the method comprising:
a) establishing nutritional ketosis in the individual;
b) determining, while the individual is experiencing nutritional ketosis, the amounts of one or more fatty acids in a biological sample from the individual to generate an optimal range of one or more fatty acids, wherein the one or more fatty acids are selected from palmitoleic acid (POA) and di-homo gamma-linolenic acid (DGLA); and
c) prescribing a diet comprising appropriate carbohydrates, or appropriate carbohydrates and proteins, such that the amounts of the one or more fatty acids are maintained within the optimal range.

2. The method of claim 1, wherein the appropriate carbohydrates comprises an appropriate amount of carbohydrates and/or an appropriate glycemic index of carbohydrates.

3. The method of claim 1, wherein the individual s at risk of developing pre-diabetes or diabetes.

4. The method of claim 1, wherein the individual has been diagnosed with pre-diabetes or diabetes.

5. The method of claim 1, wherein the individual is being treated for type-2 diabetes with a medicament.

6. The method of claim 1, wherein the individual has been diagnosed with a disorder associated with insulin resistance.

7. The method of claim 6, wherein the disorder associated with insulin resistance is selected from the group consisting of obesity, metabolic syndrome, hypertension, hepatic steatosis, polycystic ovary syndrome, and sleep apnea.

8. The method of claim 1, wherein nutritional ketosis is established when the concentration of ketones is about 0.4 mM to about 4 mM.

9. The method of claim 1, wherein nutritional ketosis is established when the concentration of ketones is about 0.5 mM to about 3 mM.

10. The method of claim 1, Wherein nutritional ketosis is established when the concentration of ketones is at least about 0.5 mM.

11. The method of claim 1, wherein nutritional ketosis is established when the concentration of ketones is at least about 0.75 mM.

12. The method of claim 1, wherein nutritional ketosis is established when the concentration of ketones is at least about 1.0 mM.

13. The method of claim 8, wherein the concentration of ketones is determined in a biological sample selected from whole blood, plasma, serum, urine, tears, and breath.

14. The method of claim 1, wherein nutritional ketosis is maintained in the individual for at least about a week before the determining step is performed.

15. The method of claim 1, wherein nutritional ketosis is maintained in the individual for at least about two weeks before the determining step is performed.

16. The method of claim 1, wherein nutritional ketosis is maintained in the individual for at least about one month before the determining step is performed.

17. The method of claim 1, wherein the at least one biological sample is a buccal swab.

18. The method of claim 1, wherein the at least one biological sample comprises cheek cells.

19. The method of claim 1, wherein the at least one biological sample is whole blood, red blood cells, plasma, or serum (e.g., serum phospholipids, serum cholesteryl esters, serum triglycerides).

20. The method of claim , wherein the determining step is qualitative.

21. The method of claim 1, further comprising determining the amounts of one or more fatty acids in a biological sample from the individual when the individual is not experiencing nutritional ketosis.

22. The method of claim 1, wherein the determining step is repeated.

23. The method of claim 1, further comprising modifying the diet of the individual so as to maintain the one or more fatty acids within the optimal range.

24. The method of claim 1, further comprising adjusting the amount and/or glycemic index of carbohydrates consumed by the individual so as to maintain the one or more fatty acids within the optimal range.

25. The method of claim 1, further comprising reducing the protein intake of the individual, if necessary, so as to maintain the one or more fatty acids within the optimal range.

26. The method of claim 1, further comprising determining a baseline of the one or more fatty acids prior to establishing nutritional ketosis.

27. The method of claim 1, further comprising analyzing serum and cheek cell data of the individual to further refine the optimum ranges for the at least one fatty acid to predict long-term weight stability,

28. A method of determining an upper limit of healthy carbohydrate intake for an individual, the method comprising:
prescribing a diet to the individual that comprises a healthy carbohydrate intake, wherein the healthy carbohydrate intake is determined for the individual by:
establishing nutritional ketosis in the individual;
determining, while the individual is experiencing nutritional ketosis, the amounts of one or more fatty acids in a biological sample from the individual to generate an optimal range of the one or more fatty acids, wherein the one or more fatty acids are selected from palmitoleic acid (POA) and di-homo gamma-linolenic acid (DGLA),
wherein the diet that comprises a healthy carbohydrate intake is a diet that maintains the amounts of the one or more fatty acids within the optimal range.

29. The method of claim 28, wherein the healthy carbohydrate intake comprises a healthy amount of carbohydrate intake and/or a healthy glycemic index of carbohydrates.
